(19) United States
(12) Patent Application Publication
(10) Pub. No.: US 2005/0147571 A1
(43) Pub. Date: Jul. 7, 2005

(54) Ultraviolet absorption

(76) Inventors: Dennis Dean Loyd, West Chester, OH (US); Larry Lee Lough, Springdale, OH (US)

Correspondence Address: Schwegman, Lundberg, Woessner & Kluth, P.A., P.O. Box 2938, Minneapolis, MN, 55402, US

(21) Appl. No.: 10/973,819

(22) Filed: Oct. 26, 2004

Related U.S. Application Data
(63) Continuation-in-part of application No. 10/701,875, filed on Nov. 5, 2003.

(51) Int. Cl. A61K 7/42

(52) U.S. Cl. 424/59; 501/33

(57) ABSTRACT

Techniques are provided for using glass as an additive to raw materials for unfinished products or as a coating for finished products. Glass is crushed into glass particles forming a glass powder. The glass powder can be mixed with raw materials in the process of making products. Alternatively, the glass powder can be mixed with liquids or gels. The resulting products that integrate the glass powder or that are coated with mixtures of the glass powder exhibit Ultra Violet (UV) absorbing properties that were originally associated with the glass and in some cases boost UV properties associated with other UV absorbing materials which may be included within the raw materials of the products.

### RELATED APPLICATIONS

The present application is a Continuation-In Part of U.S. Ser. No. 10/701,875, entitled: “Ultraviolet Absorption and Radiation Shielding for Raw Materials and Products,” filed on Nov. 5, 2003; the disclosure of which is incorporated by reference herein.

### FIELD OF THE INVENTION

The present invention is related to Ultraviolet (UV) absorbers.

### BACKGROUND OF THE INVENTION

Exiting products and coatings on products provide minimal UV production. For example, cosmetics, such as sunscreens, have a limited amount of UV production that is available to a consumer. The protection level is often dictated by introducing expensive components into the sunscreens, such as Zinc Oxide.

Other products may be coated with UV protection, via paints, sealants, and other liquids that incorporate other expensive UV absorbers. In fact, products having UV protection are often significantly more expensive to purchase than those that lack UV protection. This is due in large part to the expense associated with the materials that are conventionally thought of when providing UV protection.

Accordingly, there is a need for less-expensive additives for use with raw building materials, unfinished products, and finished products. These additives should be inexpensive vis-à-vis conventional additives and exhibit UV absorption properties that are comparable to conventional additives.

### SUMMARY OF THE INVENTION

Briefly and in general terms, glass is acquired in a powder form; the powder may include materials that are native to glass and which act as natural Ultraviolet (UV) absorbers. The powder can be mixed with raw materials of unfinished products, coated on finished products, or can be integrated with other liquids or gels. The resulting new products or newly coated products exhibit novel UV absorption properties.

### BRIEF DESCRIPTION OF THE DRAWINGS

FIG. 1 is a flowchart of a method for creating UV absorbent properties in products, according to an example embodiment.

FIG. 2 is a diagram of an UV absorbing additive, according to an example embodiment.

FIG. 3 is a diagram of an UV absorbing system, according to an example embodiment.

### DETAILED DESCRIPTION OF THE INVENTION

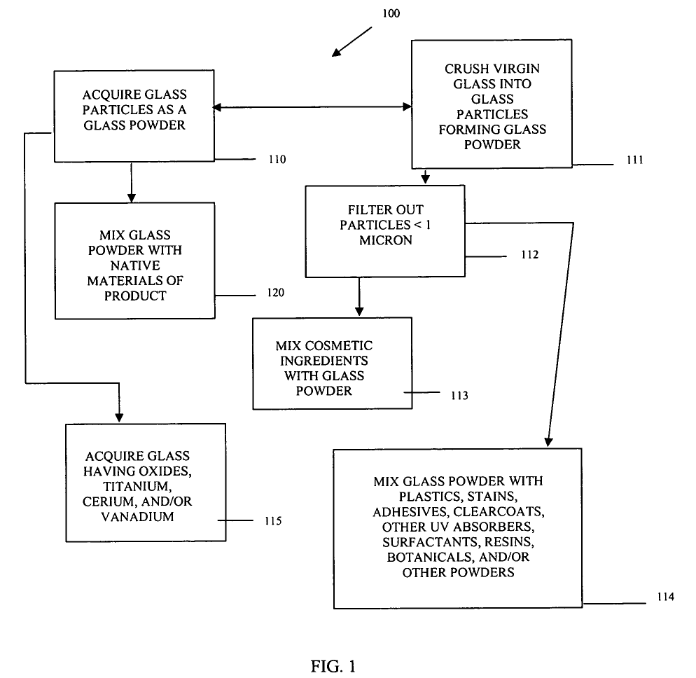

FIG. 1 illustrates a flowchart of one method 100 for creating UV absorbent properties in products. Some aspects of the method 100 may be practiced with grinding or crushing equipment and/or devices, which are adapted to grind or crush glass into small particles forming glass powders. Moreover, the method 100 is integrated with and processed in connection with devices and methods that are used to produce consumer products or to product consumer packaging. Furthermore, the method 100 can be used as a post process after finished consumer products have been produced for purposes of adding coatings to the finished products, as described herein and below.

Initially, at 110, glass is acquired. In an embodiment, the glass is acquired as virgin glass or glass specially manufactured for purposes of using the virgin glass a filler to finish products or as a coating onto finished products. For example, at 111, virgin glass is acquired and crushed into glass particles that form a glass powder. At 112, the particles of the powder are filtered out, such that each particle that remains in the glass powder has a diameter size of less than 1 micron. The powder, at 113 is then mixed with cosmetic ingredients. The result is a UV absorbent cosmetic product that absorbs UV. Moreover, the UV absorbent cosmetic product may be more cost effectively produced, since excessive amounts of Zinc oxide are not required or may be minimized within the cosmetic ingredients. Additionally, in some embodiments, the original UV absorbing ingredients which may have been included in the cosmetic's composition will have their UV absorbing properties boosted or enhanced by mixing the glass powder therewith to create the UV absorbent cosmetic product.

In an alternative embodiment, the glass particles that are approximately less than 1 micron in diameter sizes may be mixed, at 114, with plastics, stains, adhesives, clearcoats, other UV absorbers, surfactants, resins, botanicals, and/or other powders. These materials may be used to coat finished products or may be used as component materials of unfinished products.

It should be noted that the size of the glass particles that are present in the glass is configurable. Thus, in some instances where safety is less of a concern the size of the glass particles within the glass powder may be larger. Moreover, in some cases the native glass used for the glass powder may come from hazardous sources, such as from Cathode Ray Tubes (CRT) used in monitors. When hazardous glass or glass waste is used, the glass particles may be treated to remove a desired amount of hazardous lead and other heavy metals that may be present in the glass particles. Any desired level of safety can be achieved depending upon the product with which the glass powder is going to be integrated with.

Moreover, the glass can be initially acquired in a solid form or in a liquid form, such as glass slurry. Alternatively, the glass can be non hazardous such as glass containers used to hold beverages, cleaning products, household products, or food. Further, as noted above, the glass may be virgin glass that is specifically acquired or manufactured for purposes of providing glass powders as additives or coatings to products as a UV absorber.

The glass powder will include native materials originally associated with the glass from which it was derived. The small glass particles sizes may include a variety of oxides, such as titanium, cerium, zinc, other UV modifying oxides, vanadium, and others. Thus, at 115, the glass acquired has oxides or other metals that are native to the composition of the acquired glass. These oxides and perhaps other metals provide novel UV absorbent properties to the materials and ingredients with which the powder is mixed or applied. In some cases, these oxides and/or metals may also act as UV boosting agents for original materials included with the products that are UV absorbers.

At 120, the glass powder is mixed with native materials of a product. Again, the product may be something that is applied to another finished product, such as paints, clearcoats, sealants, etc. Alternatively, the native materials may be components of an unfinished product, such as plastics, metals, rubber, wood, etc. The native shape of the glass within the powder may be spheres, acicular, platelets, or various other shapes.

Conventionally, products are augmented with UV absorbers such as Zinc Oxide, Titanium Dioxide, and other additives. These conventionally preferred UV absorbers are expensive to acquire as ingredients. In fact, comparisons between lead vis-à-vis Zinc Oxide and Titanium Dioxide show that lead and other oxides or metals which are in the native composition of glass is a comparable UV absorber to these conventional absorbers. But, lead and the other modifying oxides or metals in the composition of the glass powder are more transparent than the conventional additives. Thus, the comparable level of UV absorbency is obtained with the glass powder of the present invention at a much lower production cost with increased transparency when compared to conventional absorbers. This is so, because it is less expensive to introduce a glass powder than it is to purchase and acquire conventional UV absorbers. Glass is abundant and easily acquired in virgin form or in waste form.

Therefore, the glass particles represented by the powder provides a set of unique possibilities for the economy. For example, glass waste has been conventionally disposed of or used in recycling for the primary purpose of creating recycled glass products. Conventionally, in the recycling situations, the original glass waste is restricted to non hazardous glass waste. With the teachings of this invention, what has been conventionally disregarded as waste or minimally used for recycling purposes can now be used in replace of conventional UV absorbers as additives to products. This is substantially less expensive than using convention UV absorbers and provides improved product properties, such as improved transparency.

Additionally, virgin glass designed specifically for the teachings of this invention's glass powder can be easily manufactured and acquired in desired quantities and at costs that are more desirable than conventional UV absorbing ingredients.

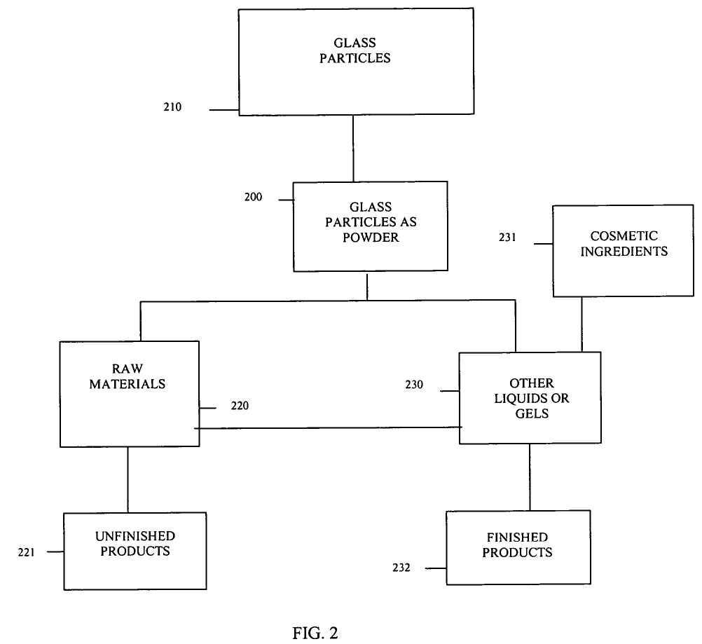

FIG. 2 is a diagram of one UV absorbing shielding additive 200. The additive 200 is a solid glass powder 200 derived from glass particles 210. The glass particles 210 have diameter sizes of less than 1 micron. In some cases, the glass powder is being mixed with cosmetic ingredients. The exact size of the glass particles 210 is configurable and can be selected based on safety considerations associated with the product being augmented with the glass powder 200.

At this small size (less than 1 micron), oxides and/or metals, such as Titanium, Cerium Oxide, Manganese Oxide, Iron Oxide, Cobalt Oxide, Copper Oxide, Vanadium Oxide, Molybdenum Oxide, Barium Oxide, and/or other salts present in the composition of the glass, which still remains in the composition of the glass are not capable of leaching from the glass particles. This is so because, at these small sizes the mass to stress equation denotes a force required for fracture to any single glass particle as being so excessive that it cannot practically be achieved in nature. This unique property of the small glass particles makes the glass particles safe for human exposure, consumption, and disposal. Again, the desired level of degree of safety can be achieved by configuring the glass particles 210 to a desired diameter size. The smaller the diameter size, the safer the powder is for consumption and exposure to humans and the environment.

In some embodiments, the glass particles 210 are derived from virgin glass that is specifically manufactured for purposes of this invention. The glass particles have diameter sizes of less than 1 micron. In a specific embodiment, the glass powder 200 that represents the glass particles 210 are adapted to be mixed into other liquids or gels 230, where those liquids or gels 230 are cosmetic ingredients 231. The resulting new modified cosmetic exhibits novel UV absorbing properties that are natively associated with the glass particles 210. Alternatively, original ingredients associated with the cosmetic 231, which are UV absorbers, have their UV absorbing properties boosted by integrating the glass powder 200 as filler. The cosmetics 231 may be associated with creams, gels, shampoo, body lotions, make-up, lipsticks, balms, sunscreens, soaps, sprays, hair care products, or any other human applied body-care product.

In other arrangements, the glass powder 200, which is derived from the glass particles, is integrated or mixed with other liquids or gels 230, such as paints, resins, stains, adhesives, clearcoats, other powders, melted plastics, plaster, other UV absorbers, fillers, surfactants, botanicals, and the like. These modified products having the glass powder 200 integrated therein can then be used as coatings on or adhesives for other finished products or building materials 232.

In still more embodiments, the glass powder 200 may be natively mixed or integrated with raw materials 220 of unfinished products 221. For example, metals, plastics, rubbers, and the like may be melted and mixed with the glass powder 200.

In some embodiments, the original glass particles 210 are associated with hazardous glass waste (e.g., CRT monitor glass and the like). In these embodiments, after the glass particles 210 are produced from the glass waste they are treated in a chemical process where they are exposed to an acid-water bath to extract any remaining surface heavy metals from the glass particles. Next, the treated glass particles are rinsed. At this point, even glass waste that was originally hazardous is now non hazardous and available for use with the teachings of the present invention.

The glass particles 210 take on a solid form as a fine-grained powder 200 at their small sizes. The powder 200 can be added to the fabrication process of unfinished products 221, where the powder 200 is mixed with raw materials 220 of the unfinished products 221. The finished products that include the powder 200 will exhibit characteristics associated with oxides and the other modifying metals found in the composition of the glass particles 210, but the harmful effects of the metals are removed because the oxides and/or metals are not capable of leaching from the product. Thus, the products will be good UV absorbers, radiation shields, and sound blockers. Moreover, the oxides and other modifying metals found in the composition of the glass particles 210 are nearly transparent, which means there is little to no detectable affect on the original color attributes associated with the products that integrate the glass particles 210.

The powder 200 can also be used to mix with other liquids or gels 230 or coatings and applied directly to finished products 232. Application can be made by spraying the finished products 232 with the powder 200 in a liquid form or by dipping the finished products 232 in a tank having the powder 200 in a liquid form. In some embodiments, application can be made by printing or rolling the liquid form of the glass powder 200 onto the product. The finished products 232 will include the beneficial properties associated with oxides and/or the other modifying metals found in the composition of the glass powder 200, but without the harmful effects normally associated with the metals, since the metals are not capable of practically leaching from the finished products 232.

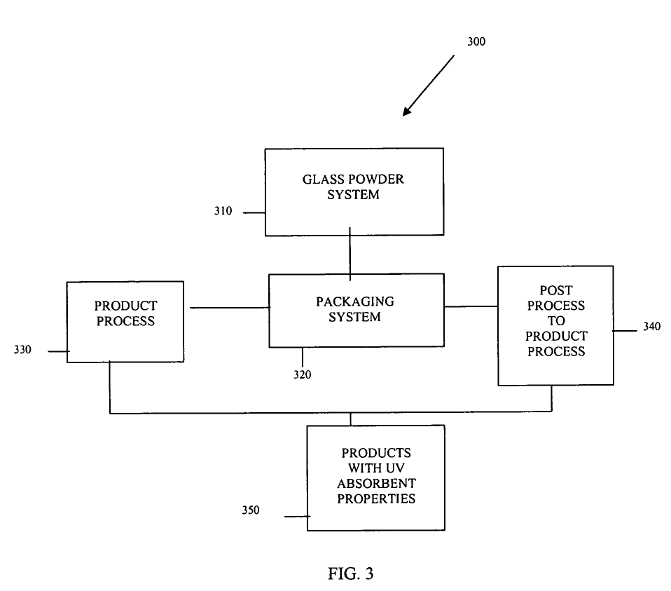

FIG. 3 is a diagram of one UV absorbing shielding system 300. The system 300 includes a glass powder system 310 and a packaging system 320. The glass powder system 310 is designed to produce glass powder from glass. The glass may be virgin glass, hazardous glass waste, or non hazardous glass waste. The glass powder system 310 is adapted to grind the glass into small glass particles. The exact diameter sizes which are associated with the glass particles are configurable, but are less than 1 micron. In embodiments, where the glass powder is being integrated into cosmetics the diameter sizes of the glass particles within the glass powder is less than 1 micron. In other embodiments, where the glass powder is being used in products that are not applied to humans or absorbed by humans through their skin, the glass particles may have slightly larger diameter sizes.

At these small and configured diameter sizes, metals and other oxides are not practically capable of leaching from the composition of the glass particles which make up the glass powder. Thus, the glass particles are safe for human exposure, consumption, and disposal.

The packaging system 320 is adapted to package the glass powder produced from the glass powder system 310 in liquid or solid forms. The solid form is a powder substance and the liquid form is the powder substance mixed with other liquids (e.g., sealants, paints, adhesives, resins, solvents, fillers, surfactants, botanicals, other UV absorbers, water, etc.). The solid form is may be used as an additive to an existing product's fabrication process 330. This performance-modifying additive is mixed with the raw materials of a product during its fabrication process 330. The result is a product 350 having embedded characteristics associated with lead and other modifying metals found in the composition of the glass particles, but without the normal harmful risks associated with oxides and/or metals that may leach from a product. These characteristics include improved UV absorption, radiation shielding, and sound blockage. In one particular embodiment, the products 350 are cosmetics, such as hair care products, gels, lotions, sunscreens, make-ups, lipstick, balms, soaps, or any other product applied onto humans.

The packaging system 320 can also produce the powder in liquid forms, which can be used in a post process to a product's fabrication process 340. The liquid forms are used to coat a finished product with the liquid. Coating can occur by brushing, spraying, rolling, printing, or dipping a finished product with the liquid. The result is a product 350 having the beneficial properties associated with oxides and/or the other modifying metals found in the composition of the glass particles, but without the harmful exposure risks which may be normally associated with oxides and/or metals, since the oxides and/or metals are not capable of practically leaching from the glass particles of the liquid.

Although specific embodiments have been illustrated and described herein, those of ordinary skill in the art will appreciate that any arrangement calculated to achieve the same purpose can be substituted for the specific embodiments shown. This disclosure is intended to cover any and all adaptations or variations of various embodiments of the invention. It is to be understood that the above description has been made in an illustrative fashion, and not a restrictive one. Combinations of the above embodiments, and other embodiments not specifically described herein will be apparent to one of ordinary skill in the art upon reviewing the above description. The scope of various embodiments of the invention includes any other applications in which the above structures and methods are used. Therefore, the scope of various embodiments of the invention should be determined with reference to the appended claims, along with the full range of equivalents to which such claims are entitled.

It is emphasized that the Abstract is provided to comply with 37 C.F.R. §1.72(b) requiring an Abstract that will allow the reader to quickly ascertain the nature and gist of the technical disclosure. It is submitted with the understanding that it will not be used to interpret or limit the scope or meaning of the claims.

In the foregoing Detailed Description, various features are grouped together in a single embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate preferred embodiment.

1. A method of adding Ultraviolet (UV) absorbent properties to products, comprising:
acquiring glass particles as a glass powder; and
mixing the glass powder with a product to create a UV absorbent product, wherein materials native to the composition of the glass particles remain in the glass powder and mix or are integrated with the product.

2. The method of claim 1 wherein acquiring further includes crushing glass into the glass particles, wherein the glass particles have diameter sizes less than 1 micron within the glass powder.

3. The method of claim 2, wherein mixing further includes mixing cosmetic ingredients with the glass powder, wherein the cosmetic ingredients are the product and the combined glass powder and cosmetic ingredients create the UV absorbent product which is a UV absorbent cosmetic product.

4. The method of claim 1 further comprising, crushing virgin glass into the glass particles that represent the glass powder.

5. The method of claim 1, wherein acquiring further includes crushing glass into the glass particles, wherein the glass particles have diameter sizes less than 1 micron and are mixed with ingredients included within a cosmetic product, and wherein the cosmetic product is at least one of a hair care product, a body or hair gel, a soap, a body lotion, a balm, a lipstick, a make-up product, and a spray product.

6. The method of claim 5, wherein mixing further includes using at least one of other UV absorbers, surfactants, resins, botanicals, and other powders as portions of the cosmetic ingredients mixed with the glass powder.

7. The method of claim 1 further comprising, acquiring the glass particles from glass having at least one of oxides, titanium, cerium, zinc, other UV modifying oxides, and vanadium.

8. An Ultraviolet (UV) absorbing additive, comprising:
a powder form of crushed glass adapted to be is used as an additive to raw materials for unfinished products or mixed with other liquids and applied to or integrated with finished products or the unfinished products, wherein the crushed glass includes materials that act as an UV absorber for the unfinished products and the finished products.

9. The UV absorbing additive of claim 8, wherein the materials include at least one of oxides, titanium, cerium, zinc, other UV modifying oxides, and vanadium.

10. The UV absorbing additive of claim 8, wherein the other liquids include gels.

11. The UV absorbing additive of claim 10, wherein the other liquids include cosmetic ingredients.

12. The UV absorbing additive of claim 10, wherein the other liquids include at least one of UV absorbers, fillers, surfactants, resins, and botanicals.

13. The UV absorbing additive of claim 10, wherein the other liquids include plastics in melted form, metals in melted form, paints, stains, adhesives, and clearcoats.

14. The UV absorbing additive of claim 8, wherein the glass powder is a fused glasseous material adapted to retain properties of the crushed glass.

15. An UV absorbing system, comprising:
a glass powder system adapted to crush glass into a glass powder; and
a packaging system to package the glass powder in a liquid or solid form and which is adapted to be mixed with materials for unfinished products or adapted to be coated onto finished products.

16. The UV absorbing system of claim 15, wherein the glass powder system is adapted to crush the glass into diameter sizes of less than 1 micron.

17. The UV absorbing system of claim 16, wherein the unfinished products are cosmetic ingredients for cosmetics, and wherein the glass powder is adapted to be incorporated into cosmetic formulations as a filler which is adapted to boost existing UV properties associated with the cosmetic ingredients.

18. The UV absorbing system of claim 15, wherein the unfinished products are in liquid or gel form when adapted to mix with the glass powder.

19. The UV absorbing system of claim 15, wherein the unfinished products at least one of plastics, metals, rubber, paints, stains, adhesives, and clearcoats.

20. The UV absorbing system of claim 15, wherein the finished products are adapted to be sprayed, dipped, rolled, or brushed with a liquid version of the glass powder.
